(19) United States
(12) Patent Application Publication
(10) Pub. No.: US 2005/0187190 A1
(43) Pub. Date: Aug. 25, 2005

(54) Autoinducer-2 compounds as immunomodulatory agents

(76) Inventors: Sanjay Vasu, Carlsbad, CA (US); Jeffrey Stein, San Diego, CA (US)

Correspondence Address: M. LISA WILSON;DUANE MORRIS LLP, 380 LEXINGTON AVENUE, NEW YORK, NY, 10168-0002, US

(21) Appl. No.: 11/040,679

(22) Filed: Jan. 21, 2005

Related U.S. Application Data
(60) Provisional application No. 60/538,890, filed on Jan. 23, 2004.

(51) Int. Cl. A61K 31/69; A61K 31/665; A61K 31/381

(52) U.S. Cl. 514/64; 514/100; 514/443

(57) ABSTRACT

The present method relates to modulating the mammalian inflammatory response using the bacterial autoinducer-2 and analogs and agonists thereof. In particular, the invention provides for ameliorating or reducing inflammation in inflammatory diseases and conditions associated with production of IL-1 and IL-6.

This application claims benefit under 35 U.S.C. § 119(e) of U.S. Provisional Ser. No. 60/538,890, filed Jan. 23, 2004, which is incorporated herein by reference.

### FIELD OF THE INVENTION

The present invention relates to modulating the mammalian inflammatory response using the bacterial autoinducer-2 and analogs and agonists thereof. In particular, the invention provides for ameliorating or reducing inflammation in inflammatory diseases and conditions associated with production of IL-1 and IL-6.

### BACKGROUND OF THE INVENTION

Inflammation, while a naturally-occurring process that protects the body against some insults, also aggravates the impact of others. Since inflammation occurs through the action of prostaglandins, previous efforts to control inflammation have employed compounds that affect prostaglandin synthesis.

Two such classes of compounds, steroids (in particular corticosteroids) and more recently non-steroidal antiinflammatory drugs (commonly known as NSAIDs), both have disadvantages. NSAIDs reduce prostaglandin-induced pain and swelling associated with inflammation but also affect other prostaglandin-regulated processes. For this reason, NSAIDs can produce severe side effects, including life-threatening ulcers, that limit their therapeutic utility. Corticosteroids, an alternative to NSAIDs, have other significant side effects. Thus, despite extensive research, a continuing need exists for a way to control inflammation while minimizing side effects.

A classic cause of inflammation results from the immune system's response to bacterial antigens produced by a bacterial infection. Expression of many of these antigens that trigger inflammation in the mammalian host is under the control of bacterial quorum-sensing, a mechanism by which bacteria coordinate expression of their genes in response to their population density. Quorum-sensing operates through signaling compounds, called autoinducers, that bacteria secrete into their surroundings. By subsequently detecting the concentrations of such compounds bacteria can determine their population density and control expression of certain genes accordingly.

Different bacteria use different autoinducers for this purpose. Gram-negative bacteria generally use an acylated homoserine lactone, termed autoinducer-1 or AI-1, as a signal, while Gram-positive bacteria use a modified oligopeptide. A recently described third system occuring in both Gram-positive and -negative bacteria uses a third class of signal, called autoinducer-2 (AI-2), based upon 3-furanone. The crystal structure of an AI-2 sensor protein, LuxP, in complex with an autoinducer (furanosyl borate diester) has been reported [Chen et al. (2002) Nature 415:488-489].

Bacterially secreted products including LPS and lipoteichoic acid stimulate an innate immune response in mammalian macrophages. This rapid cascade includes upregulation of proinflammatory cytokines IL-1 and IL-6, induction of nitric oxide synthetase (iNOS) as an antimicrobial defense, and eventual recruitment of phagocytic neutrophils in an effort to curb the incursion before infection sets in. Surprisingly, it was discovered that a functional AI-2 quorum sensing system confers ability to modulate IL-1 and IL-6 release in mouse macrophages.

### SUMMARY OF THE INVENTION

The present invention relates to methods for inhibiting inflammation, especially via inhibition of the proinflammatory response and the use thereof in the treatment of inflammatory conditions in mammals. In particular, the invention uses AI-2, AI-2 analogs or AI-2 agonists to administer to mammals in need of such treatment.

One aspect of the invention provides a method of modulating IL-1 production which comprises administering AI-2, an AI-2 analog or an AI-2 agonist to a mammal in an amount and for a time sufficient to modulate IL-1 production. Preferably IL-1 production is suppressed, in whole or in part, reduced or otherwise decreased.

Another aspect of the present invention relates to a method of modulating IL-6 production which comprises administering AI-2, an AI-2 analog or an AI-2 agonist to a mammal in an amount and for a time sufficient to modulate IL-6 production. Preferably IL-6 production is suppressed, in whole or in part, reduced or otherwise decreased.

A further aspect of the invention provides a method of treating inflammation in a mammal which comprises administering AI-2, an AI-2 analog or an AI-2 agonist to a mammal in an amount and for a time sufficient to ameliorate or reduce inflammation associated with production of IL-1 and/or IL-6. This method can be used to treat a variety of inflammatory diseases and conditions.

In each of the above methods, a naturally-occurring or enzymatically produced AI-2 is preferably used. Other preferred embodiments include the use of AI-2 analogs, including but not limited to, MHF and the oxoanion compounds described in U.S. Pat. No. 6,737,415. Pharmaceutically acceptable salts of any of these compounds can also be used. AI-2 its analogs and agonists are preferably administered as pharmaceutical compositions.

A further aspect of the invention provides a method for treating inflammation in a mammal which comprises administering autoinducer-2, an autoinducer-2 analog or an autoinducer-2 agonist to a mammal in an amount and for a time sufficient to ameliorate or reduce inflammation signalled through Toll-like receptors associated with increased iNOS activity. Preferably a naturally-occurring, enzymatically produced or chemically synthesized AI-2 is used in the method to suppress iNOS activity and concomitant accumulation of iNOS breakdown products known to stimulated the release of proinflammatory cytokines in mammals.

### BRIEF DESCRIPTION OF THE DRAWINGS

FIG. 1 is a bar graph illustrating reduction of IL-6 production by J774 macrophages infected with Lux+S. typhimurium at a multiplicity of infection (MOI) of 0.1:1 relative to the same macrophages infected with Lux− S. typhimurium at the same MOI. Secreted IL-6 in macrophage culture supernatants was measured by ELISA.

FIGS. 2A and B are bar graphs illustrating that AI-2 is sufficient to produce the anti-inflammatory effect in J774 macrophages in the absence of bacteria. FIG. 2A shows, from left to right, IL-6 production after (1) No treatment; (2) treatment with LPS alone; (3) concurrent treatment with LPS and sterile supernatant from an S. typhimurium LuxS− strain; and (4) concurrent treatment with LPS and sterile supernatant from an S. typhimurium LuxS+ strain. FIG. 2B shows IL-6 production after treatment with LPS and ribose (a compound structurally similar to AI-2 but which is inactive in quorum sensing)(left) and after concurrent treatment with LPS and enzymatically-synthesized AI-2 (right). Secreted IL-6 measured as in FIG. 1.

FIG. 3 is a bar graph illustrating suppression of IL-1 and IL-6 production by AI-2 on LPS-stimulated J774 macrophages. “No LPS” represents production in the absence of LPS stimulation; “no AI-2” represents production after LPS stimulation but no AI-2; and “10 ul AI-2” represents production with LPS stimulation and concurrent exposure to AI-2 (40 μM). Secreted IL-1 (light bars) and IL-6 (dark bars) in macrophage culture supernatants was measured by ELISA.

FIG. 4A and FIG. 4B are bar graphs illustrating increasing suppression of IL-1 (A) and IL-6 (B) production by increasing amounts of enzymatically-prepared AI-2 on LPS-stimulated macrophages. Secreted IL-1 and IL-6 was measured as in FIG. 3.

FIG. 5A is a bar graph illustrating suppression of IL-6 production by titration with the AI-2 analog 5-methyl-4-hydroxy-3(2H)furanone (MHF) on stimulated J774 macrophages. Cell metabolic activity is also indicated. FIG. 5B is bar graph illustrating that 500 μg/ml MHF achieves quorum-sensing activity comparable to 2.5 μL enzymatically-synthesized AI-2 and that 50 μM ribose lacks quorum-sensing activity. MHF is indicated as Compound 34 on these graphs.

FIG. 6 is a bar graph illustrating suppression of IL-6 production by the AI-2 analog MHF on viral ribopolymer (polyCI)-stimulated J774 macrophages. “None” represents production in the absence of any treatment; “polyCI” represents production after polyCI stimulation but in the absence of MHF; “polyCI+AI-2 analog” represents production with polyCI stimulation and concurrent exposure to MHF; “polyCI+ribose” represents production with polycI stimulation and concurrent exposure to ribose. Secreted IL-6 measured as in FIG. 1; replicate experiments shown.

FIG. 7 is a bar graph illustrating suppression of IL-6 production by the AI-2 analog MHF on lipoteichoic acid-stimulated J774 macrophages. “No Treat” represents production in the absence of any treatment; “Lipo+Ribose” represents production with lipoteichoic acid stimulation and concurrent exposure to ribose; and “Lipo+AI-2 analog” represents production with lipoteichoic stimulation and concurrent exposure to MHF. Secreted IL-6 measured as in FIG. 1; replicate experiments shown.

FIG. 8 is a bar graph illustrating suppression of IL-6 production by the AI-2 analog MHF on LPS-stimulated J774 macrophages. “None” represents production in the absence of any treatment; “LPS” represents production after LPS stimulation but in the absence of MHF; “LPS+AI-2 analog” represents production with LPS stimulation and concurrent exposure to MHF; “LPS+ribose” represents production with LPS stimulation and concurrent exposure to ribose. Secreted IL-6 measured as in FIG. 1; replicate experiments shown.

FIG. 9 is a bar graph illustrating inhibition of Cox-2 prostaglandin synthetase activity by AI-2 and the AI-2 analog MHF. “Max Inhibition” and “Aspirin/1000 uM” are positive inhibition controls. “Analog/500 uM,” represents the inhibition of prostaglandin synthesis by 500 μM MHF; “AI-2/40 uM” and “AI-2/80 uM” represents the inhibition of prostaglandin synthesis by 40 and 80 μM, respectively of AI-2. “Ribose/500 um” the inhibition of prostaglandin synthesis by 500 μM ribose. Prostaglandin synthesis was indirectly quantitated using an ELISA for PGE₂ production.

FIG. 10 is a bar graph showing that AI-2 treatment suppresses nitric oxide production by LPS-stimulated J774 macrophages. “LPS+34,” “LPS+AI-2(5 ul)” and LPS+AI-2(2 ul) represents inhibition by concurrent treatment with LPS and MHF or the indicated amounts of AI-2. “LPS+ribose” is a control to show the amount of nitric oxide production in the absence of an inhibitor.

### DETAILED DESCRIPTION OF THE INVENTION

As indicated, AI-2 is not only involved in signaling between bacteria, but surprisingly also influences the immune system of mammalian hosts. While previous studies showed that N-(3-oxohexanoyl)-L-homoserine lactone, an AI-1 from Pseudomonas aeruginosa, stimulates the mammalian immune system, the impact of bacterial AI-2 upon the mammalian immune system was unsuspected in view of the different chemical structures of the two classes of autoinducers.

In particular, AI-2 (as well as agonists and analogs thereof) reduces production of the proinflammatory cytokines, IL-1 and IL-6 from mouse macrophages. These cytokines are associated with the initial innate immune response to markers of bacterial presence, including bacterial lipopolysaccharides and exotoxins.

AI-2 therefore acts not only as a quorum-sensing signal in bacteria but as an immunosuppressant in mammals (by directly targeting and regulating mammalian immune mechanisms). For example, AI-2 and its analogs/agonists (i.e., compounds that, like AI-2, activate the quorum-sensing systems of bacteria) act directly on mammalian cells to attenuate release of the proinflammatory cytokines IL-1 and IL-6 in the absence of bacteria.

This discovery provides a way to control the immune response, and in particular to reduce inflammation, by administering to a patient in need thereof an efficacious amount of AI-2 or an AI-2 agonist/analog. Anti-inflammatory agents based upon use of AI-2, its agonists and analogs would allow treatment of inflammation without the adverse side-effects associated with the steroids and NSAIDs currently used for this purpose.

Accordingly, the present invention provides a method of treating inflammation in a mammal which comprises administering AI-2, an AI-2 analog or an AI-2 agonist to a mammal in an amount and for a time sufficient to ameliorate or reduce inflammation associated with production of IL-1 and/or IL-6.

AI-2 can be naturally occurring from a bacterial source known to produce AI-2 or molecules with AI-2 activity (i.e., analogs or agonists) as well as enzymatically produced AI-2.

Naturally-occurring AI-2 can be purified from the native source using conventional purification techniques, derived synthetically by chemical means, or preferably, produced by the in vitro method described in U.S. Pat. No. 6,780,890.

“Autoinducer-2 analog,” “AI-2 analog” or “AI-2 agonist” means any compound with at least 10% of the autoinducer-2 activity of any stereoisomer of 4-hydroxy-5-methyl-2H-furan-3-one, and includes the naturally-occurring AI-2.

Such agonists and analogs can be readily recognized by their action on the quorum-sensing system of bacteria such as mutant strains of Vibrio harveyi that respond to AI-2 but cannot produce it themselves. For example, AI-2 analogs and agonists can be identified by known techniques, including but not limited to, large-scale screening of compounds through use of the V. harveyi bioassay described in U.S. Pat. No. 6,780,890 and other techniques described therein. Reduction in signaling activity in the presence of a test compound indicates the ability of that compound to, for example, block bacterial pathogenesis by affecting the expression of one or more virulence factors.

Examples of AI-2 analogs suitable for the present invention, include but are not limited to, the AI-2 analog 5-methyl-4-hydroxy-3(2H)furanone (MHF) as well as the compounds represented by structure I 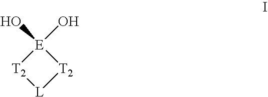
wherein E is selected from the group consisting of B, P, and S;
T₁, and T₂ are each independently selected from the group consisting of O, NR, and CH₂, where R═H or C₁-C₈ alkyl, or C₁-C₈ oxoalkyl; and

L is selected from the group consisting of ethylene, propylene, and four to six-membered alicyclic and aromatic rings. Preferably, E is B (boron) or P (phosphorous). Preferably, T₁, and T₂ are O (oxygen). Preferably, the compound has a molecular weight less than about 750 Da, more preferably, less than about 500 Da.

In accordance with the invention, L groups include ethylene, propylene, cyclopentyl, cyclohexyl, pyrrolidine, tetrahydrofuran, piperidine, pyran, dioxane, morpholine, pyrrole, furan, pyridine, pyrimidine, pyrazine, imidazole, thiazole, oxazole, purine, and indazole. Particularly preferred L groups include ethylene, propylene, cyclopentyl, cyclohexyl, pyrrolidine, tetra-hydrofuran, piperidine, pyran, dioxane, and morpholine. Most preferred L groups include cyclopentyl, cyclohexyl, pyrrolidine, tetrahydrofuran, piperidine, pyran, dioxane, and morpholine.

In another preferred embodiment, L is tetrahydrofuran bearing a keto, a hydroxy, and a carboxamido functional group, T₁ and T₂ are oxygen, and E is B or P. More preferably, the compound has the following structure:

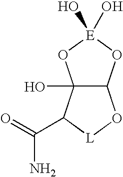

The AI-2 analogs represented by structure I are described in U.S. Pat. No. 6,737,415, which also includes methods of synthesizing those compounds. Reference to a particular compound herein is to be understood as a reference to the compound itself and any salts thereof, and vice versa. Compounds that possess an acidic or basic group may form pharmaceutically-acceptable salts with pharmaceutically-acceptable cations or anions. Examples of pharmaceutically-acceptable cations include ammonium, tetramethylammonium, alkali metal (e.g. sodium, lithium and potassium) and alkaline earth metal (e.g. calcium, barium and magnesium), aluminum, zinc, and bismuth cations, and protonated forms of basic amino acids, such as arginine, lysine, and organic amines such as ethanolamine, ethylenediamine, triethanoleamine, benzylphenethylamine, methylamine, dimethylamine, trimethylamine, diethylamine, piperidine, morpholine, tris-(2-hydroxyethyl)amine, and piperazine.

Examples of pharmaceutically-acceptable anions include those derived from inorganic acids such as hydrochloric, hydrobromic, hydriodic, sulfuric, and phosphoric acid, as well as organic acids such as p-toluenesulfonic, methanesulfonic, oxalic, p-bromo-phenylsulfonic, carbonic, succinic, citric, benzoic, and acetic acid, and related inorganic and organic acids. Such pharmaceutically-acceptable salts include sulfate, pyrosulfate, bisulfate, sulfite, bisulfite, phosphate, ammonium, monohydrogenphosphate, dihydrogenphosphate, meta-phosphate, pyrophosphate, chloride, bromide, iodide, acetate, propionate, decanoate, caprylate, acrylate, formate, isobutyrate, caprate, heptanoate, propiolate, oxalate, malonate, succinate, suberate, hippurate, butyne-1,4-dioate, hexane-1,6-diospate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, alpha-hydroxybutyrate, glycolate, maleate, tartrate, methanesulfonate, propanesulfonate, naphthalene-1-sulfonate, naphthalene-2-sulfonate, mandelate. It is understood that the above salts may form hydrates or exist in a substantially anhydrous form.

The present invention is useful to modulate inflammation and treat an inflammatory disease or condition disorder in a mammal.

As used herein, an “inflammatory disease or condition disorder” is intented to include a disease or condition characterized by, caused by, resulting from, or becoming affected by inflammation. Examples of inflammatory diseases or conditions include, but are not limited to, acute and chronic inflammation disorders such as asthma, psoriasis, rheumatoid arthritis, osteoarthritis, psoriatic arthritis, inflammatory bowel disease (Crohn's disease, ulcerative colitis), sepsis, vasculitis, and bursitis; autoimmune diseases such as Lupus, Polymyalgia, Rheumatica, Scleroderma, Wegener's granulomatosis, temporal arteritis, cryoglobulinemia, and multiple sclerosis; transplant rejection; osteoporosis; cancer, including solid tumors (e.g., lung, CNS, colon, kidney, and pancreas); Alzheimer's disease; atherosclerosis; viral (e.g., HIV or influenza) infections; chronic viral (e.g., Epstein-Barr, cytomegalovirus, herpes simplex virus) infection; and ataxia telangiectasia.

Pathological processes associated with a pro-inflammatory response in which the AI-2, its analogs and agonists are useful for treatment including, but are not limited to, asthma, allergies such as allergic rhinitis, uticaria, anaphylaxis, drug sensitivity, food sensitivity and the like; cutaneous inflammation such as dermatitis, eczema, psorisis, contact dermatitis, sunburn, aging, and the like; arthritis such as osteoarthritis, psoriatic arthritis, lupus, spondylarthritis and the like. AI-2, its analogs and agonists are also useful for treating chronic obstruction pulmonary disease and chronic inflammatory bowel disease. The AI-2, its analogs and agonists may further be used to replace corticosteroids in any application in which corticosteroids are used including immunosuppression in transplants and cancer therapy.

As used herein, mammals include humans and domesticated animals. In a preferred embodiment, the mammal is a primate. In an even more preferred embodiment, the primate is a human.

As used herein, the term “administering” to a mammal includes dispensing, delivering or applying a compound of the invention, e.g., in a pharmaceutical formulation (as described herein), to a mammal by any suitable route for delivery of the compound to the desired location in the subject, including delivery by either the parenteral or oral route, intramuscular injection, subcutaneous/intradermal injection, intravenous injection, buccal administration, transdermal delivery and administration by the rectal, colonic, vaginal, intranasal or respiratory tract route (e.g., by inhalation).

As used herein, the term “an amount sufficient” or an “effective amount” includes an amount effective, at dosages and for periods of time necessary, to achieve the desired result, e.g., sufficient to treat the inflammatory disease or condition in a mammal. An effective amount of the compounds of the invention, as defined herein may vary according to factors such as the disease state, age, and weight of the mammal, and the ability of the compound to elicit a desired response in the mammal. Dosage regimens can be adjusted to provide the optimum therapeutic response. An effective amount is also one in which any toxic or detrimental effects (e.g., side effects) of the compound are outweighed by the therapeutically beneficial effects.

A therapeutically-effective amount of AI-2, its analogs and agonists (i.e., an effective dosage) may range from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan appreciates that certain factors influence the dosage required to effectively treat a mammal (and thereby ameliorate or reduce the inflammation), including but not limited to the severity of the disease or condition, previous treatments, the general health and/or age of the mammal, and other diseases present. Moreover, treatment of a mammal with a therapeutically-effective amount of AI-2, its analogs or agonists can include a single treatment or, preferably, can include a series of treatments. In one example, a mamma; is treated with a compound of the invention in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks.

AI-2, its analogs or agonists can be provided alone, or in combination with other agents that modulate a particular pathological process. For example, these compounds can be administered in combination with other known anti-inflammatory agents. Known anti-inflammatory agents that may be used in the methods of the invention can be found in Harrison's Principles of Internal Medicine, Thirteenth Edition, Eds. T. R. Harrison et al. McGraw-Hill N.Y., N.Y.; and the Physicians Desk Reference 50th Edition 1997, Oradell N.J., Medical Economics Co., the complete contents of which are expressly incorporated herein by reference.

Examples of other anti-inflammatory agents that can be used in conjunction with AI-2, its analogs and agonists include monoclonal antibodies directed against TNF-α (e.g., Rituxan) or monoclonal antibodies that block the activity of other inflammation inducing proteins (e.g., other cytokines or interleukins). Monoclonals, which bind irreversibly to their target, are know to increase susceptabiltiy to infection due to the long term attenuation of the inflammatory response. AI-2, its analogs and agonist may lower the amount of other drugs needed and, as a small molecule drug, may not not bind irreversibly.

AI-2, its analog and agonists and the additional anti-inflammatory agents can be administered to the mammal in the same pharmaceutical composition or in different pharmaceutical compositions (at the same time or at different times).

Pharmaceutical Preparations

AI-2, its analogs and agonists can be formulated as pharmaceutical compositions comprising one or more of those molecules together with a pharmaceutically acceptable carrier. Pharmaceutically acceptable carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical carriers are described in Gennaro et al., (1995) Remington's Pharmaceutical Sciences, Mack Publishing Company. In addition to the pharmacologically active agent, the compositions can contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds, as appropriate in oily injection suspensions may be administered. Suitable lipophilic solvents or vehicles include fatty oils, for example, sesame oil or synthetic fatty acid esters, for example, ethyl oleate or triglycerides. Aqueous injection suspensions can contain substances which increase the viscosity of the suspension include, for example, sodium carboxymethyl cellulose, sorbitol, and dextran. Optionally, the suspension can also contain stabilizers. Liposomes can also be used to encapsulate the agent for delivery into the cell.

The pharmaceutical formulation for systemic administration according to the invention can be formulated for enteral, parenteral or topical administration. Indeed, all three types of formulations can be used simultaneously to achieve systemic administration of the active ingredient.

Suitable formulations for oral administration include hard or soft gelatin capsules, pills, tablets, including coated tablets, elixirs, suspensions, syrups or inhalations and controlled release forms thereof.

AI-2, its analogs and agonists can also be incorporated into pharmaceutical compositions which allow for the sustained delivery of those compounds to a mammal for a period of several days, to at least several weeks, to a month or more. Such formulations are described in U.S. Pat. Nos. 5,968,895 and 6,180,608 B1.

For topical administration, any common topical formation such as a solution, suspension, gel, ointment or salve and the like can be employed. Preparation of such topical formulations are well described in the art of pharmaceutical formulations as exemplified, for example, by Remington's Pharmaceutical Sciences. For topical application, AI-2, its analogs and agonists can also be administered as a powder or spray, particularly in aerosol form. The active ingredient can be administered in pharmaceutical compositions adapted for systemic administration. As is known, if a drug is to be administered systemically, it can be confected as a powder, pill, tablet or the like or as a syrup or elixir for oral administration. For intravenous, intraperitoneal or intra-lesional administration, the active ingredient will be prepared as a solution or suspension capable of being administered by injection. In certain cases, it may be useful to formulate the active ingredient in suppository form or as an extended release formulation for deposit under the skin or intramuscular injection. In a preferred embodiment, AI-2, its analogs and agonists can be administered by inhalation. For inhalation therapy the compound can be in a solution useful for administration by metered dose inhalers or in a form suitable for a dry powder inhaler.

It will be appreciated by those skilled in the art that various omissions, additions and modifications may be made to the invention described above without departing from the scope of the invention, and all such modifications and changes are intended to fall within the scope of the invention, as defined by the appended claims. All references, patents, patent applications or other documents cited are herein incorporated by reference.

### EXAMPLE 1

### Materials and Methods

Tissue Culture and Immunostimulation of Cells

J774.1 macrophages were cultured in RPMI 1640 medium containing 15% fetal calf serum in a humidified 37° C. incubator supplied with 5% CO₂. Cells were plated at 50% confluency in 96 well format and allowed to attach overnight. For immunostimulation experiments, cells were incubated overnight in 100 μl of the above medium containing sub-maximal stimulatory levels of inducers: 0.1 μg/mL Salmonella lipopolysaccharide (Sigma), 1 μg/mL lipoteichoic acid (Sigma) or sterile, cell-free bacterially conditioned medium at a final concentration of 10⁷ cell equivalents/mL. Following incubation, the medium was removed, centrifuged, and stored at −20° C. for subsequent assay for the presence of released cytokines. Preparation of bacterially conditioned medium Pseudomonas aeruginosa strain PAO1 and Lux⁺ or Lux⁻ strains of Salmonella typhimurium 14028 were cultured in Luria Broth overnight. The optical density of the cultures was measured at 600 nm and cell densities calculated through use of a MacFarland's standard. Bacterial cultures were centrifuged and supernatants were filtered through a 0.2 micron sterile filter to remove all bacteria.

Invasion Assay

Salmonella typhimurium LuxS⁺ or LuxS⁻ strains were centrifuged at 500 g for 5 minutes onto J774 macrophages in serum-free Roswell Park Memorial Institute (RPMI) 1640 medium at a multiplicity of infection of 10 mammalian cells to every bacterium. Invasion was allowed to proceed for 1 hour at which point medium and non-invaded bacteria were removed, the macrophage monolayer washed and the medium replaced with RPMI 1640/15% calf serum/S0 μg/mL amikacin. Cells were allowed to recover for 2 hours. Killed bacteria and drugged medium were removed and replaced with fresh medium containing amikacin. Invaded cells were washed again to remove any remaining debris or loose cells and incubated overnight in fresh medium containing amikacin.

ELISA assay for the Release of Proinflammatory Cytokines

Centrifuged macrophage culture supernatants were assayed for the presence of secreted IL-1 and IL-6 through use of an ELISA kit according to the manufacturer's directions (R&D Systems, Minneapolis, Minn.). Briefly, 10 μl of culture supernatants were bound to test wells precoated with either anti-IL-1 or anti-IL-6 immunoglobulin. Bound cytokine was detected through use of a secondary cytokine-specific antibody tagged with horseradish peroxidase. Assay samples were developed through use of stabilized tetramethylbenzidine and hydrogen peroxide. Cytokine level, directly proportional to the colorimetric signal, was quantitated by measuring sample absorbance at 450 nm.

Cytotoxicity Assay

Cytotoxicity of immunostimulatory treatments was monitored using either an Alamar Blue metabolic activity assay directly on the treated cells or a CytoTox96 lactate dehydrogenase (LDH) assay kit (Promega). Briefly, for the Alamar Blue assay, medium was removed from the cells and replaced with fresh medium containing 0.2 mg/mL resazurin dye. Cells were incubated for 3 hours in a humidified 37° C. incubator supplied with 5% CO₂. Dye reduction, an indication of cellular metabolic activity, was measured by excitation of the resazurin at 530 nm and measurement of the fluorescence emitted at 590 nm. Release of lactate dehydrogenase was used as measure of cellular lysis and death. The LDH assay was performed on 10 μl aliquots of macrophage culture supernatants according to the manufacturer's directions.

AI-2 Synthesis In Vitro

AI-2 was synthesized as previously described (Schauder, S., Shokat, K., Surette, M. G., and Bassler, B. L. (2001) Mol. Microbiol. 41, 463-476.). Briefly, S-adenosylhomocysteine was converted to S-ribosylhomocysteine by incubation with 100 μg/mL recombinant Pfs enzyme in 50 mM Tris pH 7.6. S-ribosylhomocysteine was converted to AI-2 by 100 μg/mL recombinant LuxS. Proteins were removed from the preparation by ultrafiltration through a millipore microcentrifuge filter unit with a 5 kD cutoff. AI-2 was quantitated by measuring homocysteine, a co-product of the LuxS reaction produced in stoichiometric proportion to AI-2 in the LuxS reaction. Homocysteine concentration was determined by A_{412 nm} in the presence of 2.5 mM 5,5′-dithiobis(2-nitrobenzoic acid) (Ellman's reagent).

### EXAMPLE 2

### A Functional luxS Gene Confers Ability to Attenuate IL-1 and IL-6 Production

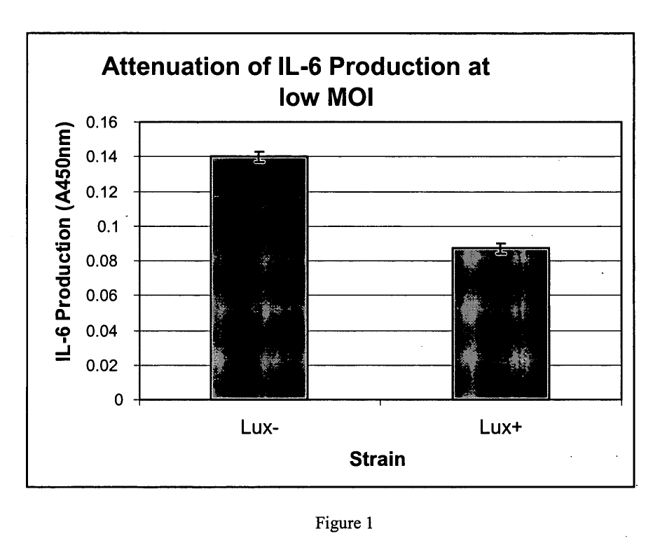

LuxS⁺ and LuxS⁻ strains of Salmonella typhimurium 14028 differ little in intracellular bacterial counts and hence in invasion phenotypes. At an multiplicity of infection of 100:1, macrophages harboring invading bacteria show a strong proinflammatory cytokine response, independent of LuxS genotype. However, at lower multiplicity of infection values, the LuxS+ strain exhibits reduced stimulation of both IL-1 and IL-6. Under these conditions, stimulation of these proinflammatory cytokines occurs only at 20 hours post invasion. At this time a differential between the ability of LuxS⁺ and LUXS⁻ to impact the IL-1 and IL-6 production occurs at an multiplicity of infection of 0.1:1 (FIG. 1).

### EXAMPLE 3

### AI-2 is Sufficient to Produce the Anti-Inflammatory Effect in J774 Macrophages in the Absence of Bacteria

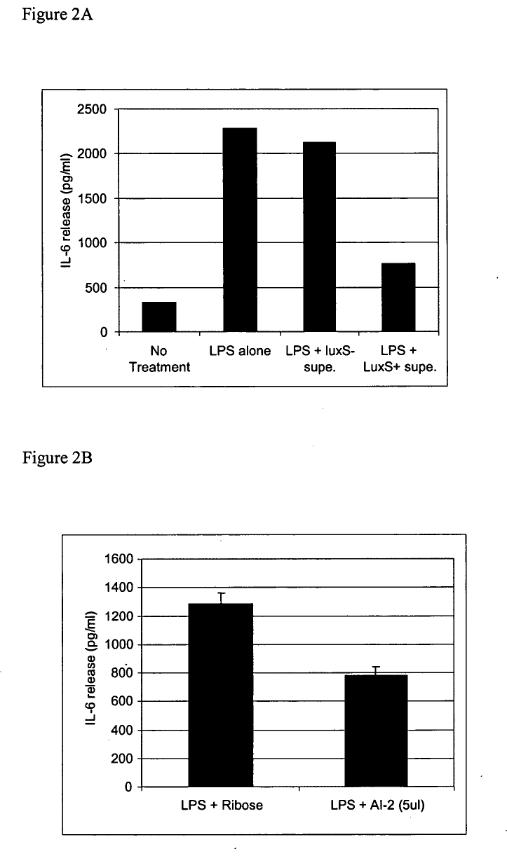

The proinflammatory response stimulated by Pseudomonas aeruginosa LPS (0.1 μg/ml) does not require the physical presence of bacteria or direct interaction between bacteria and mammalian cells since the sterile supernatant from Lux+bacteria is sufficient to reduce the IL-6 response (FIG. 2A). To test whether the product of LuxS is responsible for the anti-inflammatory effect, we synthesized AI-2 enzymatically. Macrophages stimulated with LPS and then treated with 5 μl of the AI-2 reaction product exhibit almost 50% reduction in IL-6 levels (FIG. 2B).

### EXAMPLE 4

### AI-2 Anti-Inflammatory Activity does not Depend on Stimulus

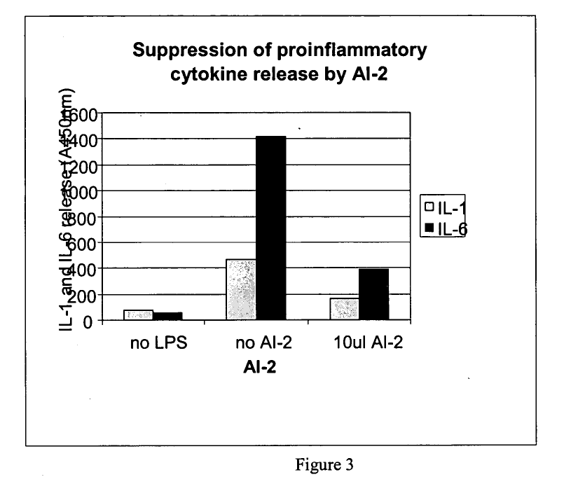

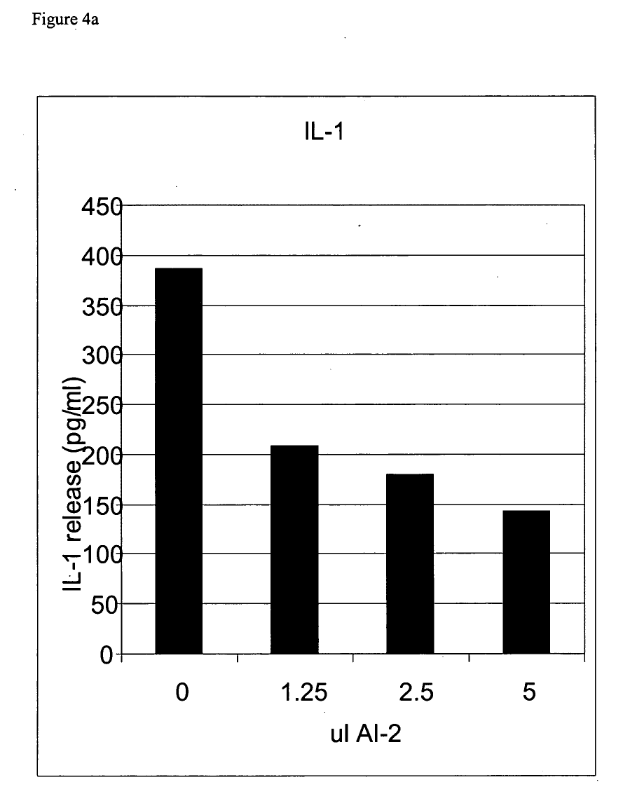

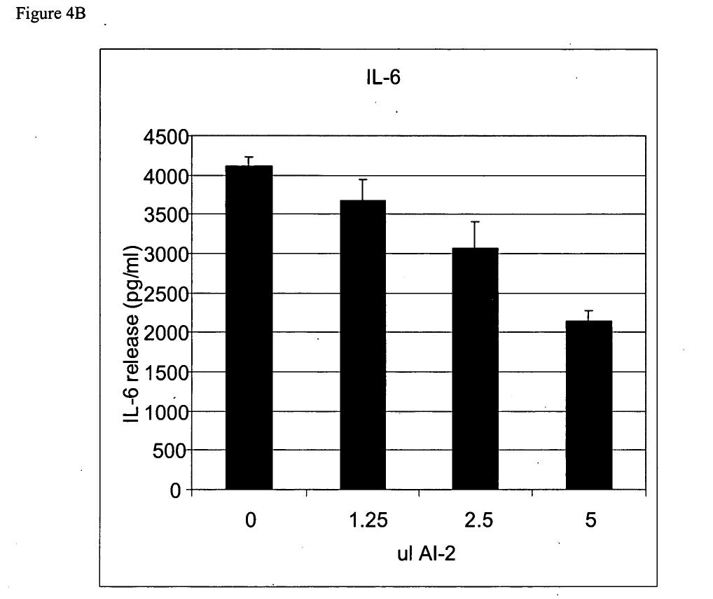

AI-2 and analogs attenuate macrophage production of proinflammatory cytokines in the presence of bacterially-produced stimulatory compounds. Lipopolysaccharide (LPS; 0.1 μg/mL) generates strong but sub-maximal stimulation of IL-1 and IL-6 production. Concurrent exposure of LPS-treated macrophages to authentic AI-2 (40 μM) or analogs (at 877 μM) reduces the overall concentration of both IL-1 and IL-6 by approximately 50% (FIG. 3 and FIG. 4). In contrast, compounds structurally resembling AI-2 (e.g., ribose and ascorbic acid) but lacking quorum sensing activity affect neither IL-1 nor IL-6 production. In addition, in the absence of exogenously-supplied immunostimulatory factors neither AI-2 nor its biosynthesis co-products, adenine and homocysteine, cause macrophages to release IL-1 or IL-6.

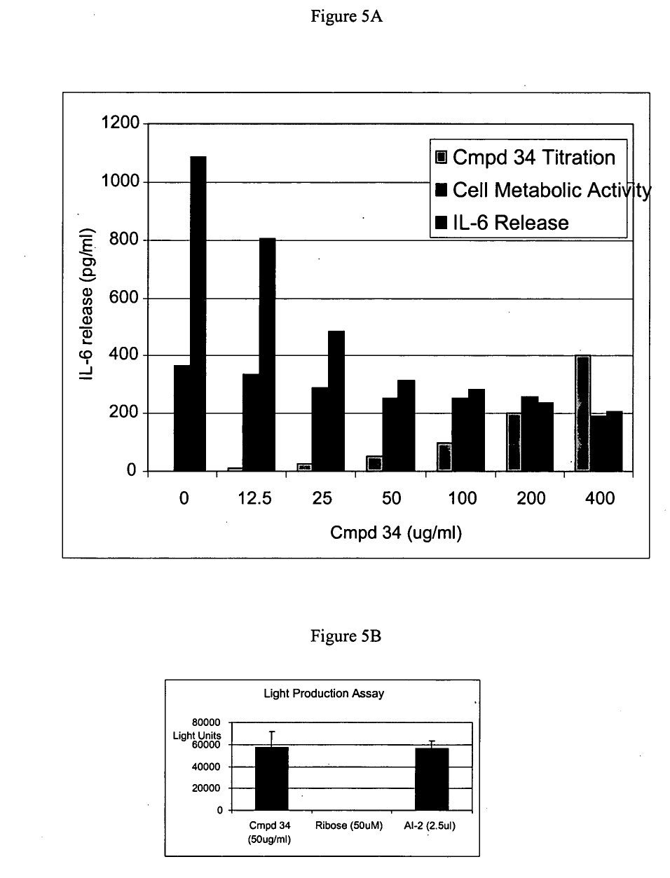

Attentuation of IL-6 release by the MHF displays dose-dependent kinetics with detectable attenuation occurring at 105 μM and 50% reduction in IL-6 production at 500 μM MHF (FIG. 5A). Near maximal attenuation occurs at 877 μM with little change in attenuation occurring at higher concentrations. This saturable response suggests interaction between MHF and a specific target.

MHF displays quorum sensing activity in the MM32 light production assay (FIG. 5B).

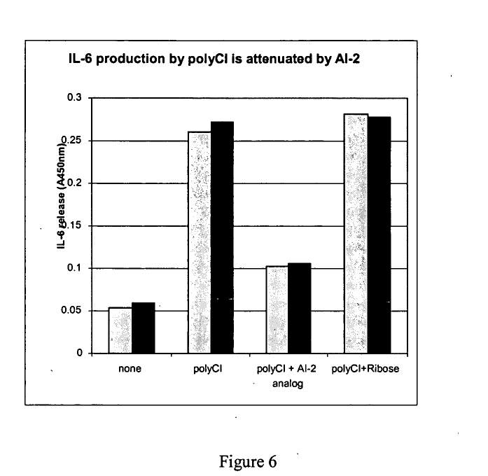

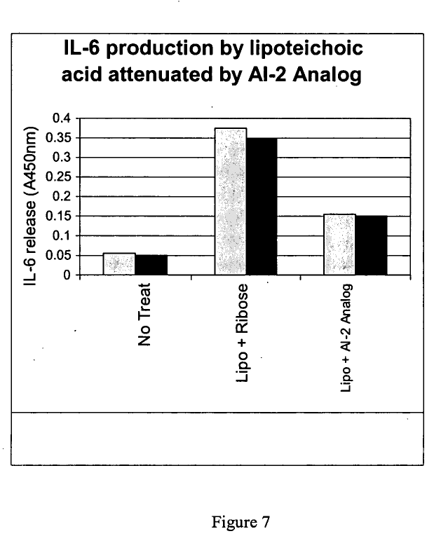

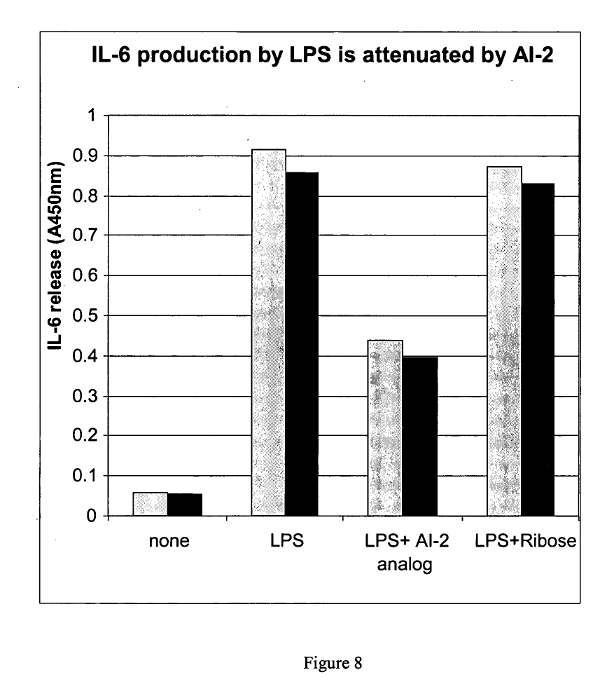

AI-2 and its analog MHF decrease production of IL-6 regardless of whether IL-6 production is stimulated by viral ribopolymers, LPS or lipoteichoic acid (a Gram-positive cell wall component). Ribose, which structurally but not functionally resembles AI-2 effects no attenuation (FIGS. 6-8). Thus suppression of proinflammatory cytokine levels in stimulated macrophages by AI-2 and MHF does not depend on the type of stimulus, consistent with their acting on a downstream signaling or regulatory step.

### EXAMPLE 5

### AI-2 Inhibits COX-2 In Vitro

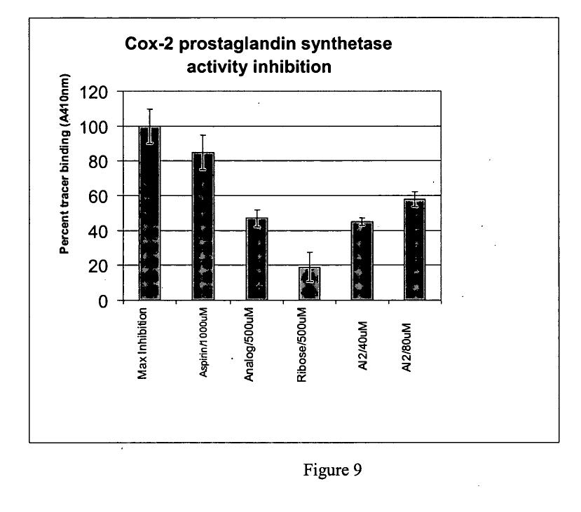

Extracellular bacterial signals trigger expression of cyclooxygenase-2 (cox-2), which amplifies the cellular IL-6 response by catalyzing production of prostaglandin PGE₂. AI-2 reduces the prostaglandin level produced by recombinant COX-2 in a dose-dependent fashion with an IC₅₀ of 40 μM, the same IC₅₀ value with which AI-2 inhibits IL-6 production in macrophages (FIG. 9), and comparable with the IC₅₀ values of known COX-2 inhibitors. This finding, along with the inability of ribose (even at high concentrations, 500 μM) to inhibit PGE₂ production, points to a specific interaction between AI-2 and COX-2.

Aspirin, a non-specific inhibitor of COX-2 served as an inhibition control; prostaglandin synthesis was indirectly quantitated in an ELISA assay.

### EXAMPLE 6

### AI-2 Treatment Suppresses Nitric Oxide Production by LPS Stimulated Macrophages

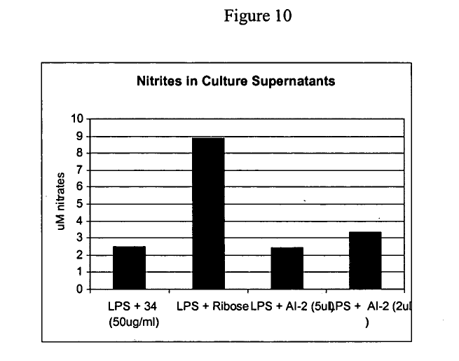

Nitric oxide production, an antimicrobial response to infectious agents, is highly induced (via iNOS activity) in macrophages exposed to LPS. Treatment of macrophages with AI-2 or compound 34 reduces nitrite (one of two spontaneous breakdown products of nitric oxide) to near baseline levels (FIG. 10).

1. A method of modulating IL-1 production which comprises administering autoinducer-2, an autoinducer-2 analog and/or anautoinducer-2 agonist to a mammal in an amount and for a time sufficient to modulate IL-1 production.

2. The method of claim 1, wherein autoinducer-2 is administered.

3. The method of claim 1, wherein said analog is 5-methyl-4-hydroxy-3(2H)furanone.

4. The method of claim 1, wherein said analog is a compound of the formula 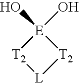
wherein E is selected from the group consisting of B, P, and S;
T₁, and T₂ are each independently selected from the group consisting of O, NR, and CH₂, where R═H or C₁-C₈ alkyl, or C₁-C₈ oxoalkyl; and
L is selected from the group consisting of ethylene, propylene, and four to six-membered alicyclic and aromatic rings;
or a pharmaceutically acceptable salt thereof.

5. The method of claim 4, wherein E is B or P; T₁ and T₂ are 0 and L is tetrahydrofuran group bearing a keto, a hydroxy, and a carboxamido functional group.

6. The method of claim 5 wherein said compound is represented by the formula

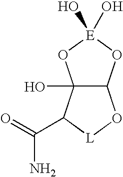

7. A method of modulating IL-6 production which comprises administering autoinducer-2, an autoinducer-2 analog and/or anautoinducer-2 agonist to a mammal in an amount and for a time sufficient to modulate IL-6 production.

8. The method of claim 6, wherein autoinducer-2 is administered.

9. The method of claim 6, wherein said analog is 5-methyl-4-hydroxy-3(2H)furanone.

10. The method of claim 6, wherein said analog is a compound of the formula 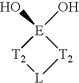
wherein E is selected from the group consisting of B, P, and S;
T₁, and T₂ are each independently selected from the group consisting of O, NR, and CH₂, where R═H or C₁-C₈ alkyl, or C₁-C₈ oxoalkyl; and
L is selected from the group consisting of ethylene, propylene, and four to six-membered alicyclic and aromatic rings;
or a pharmaceutically acceptable salt thereof.

11. The method of claim 10, wherein E is B or P; T₁ and T₂ are 0 and L is tetrahydrofuran group bearing a keto, a hydroxy, and a carboxamido functional group.

12. The method of claim 11 wherein said compound is represented by the formula

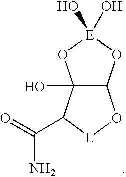

13. A method of treating inflammation in a mammal which comprises administering autoinducer-2, an autoinducer-2 analog or an autoinducer-2 agonist to a mammal in an amount and for a time sufficient to ameliorate or reduce inflammation associated with production of IL-1 and/or IL-6.

14. The method of claim 13, wherein autoinducer-2 is administered.

15. The method of claim 13, wherein said analog is 5-methyl-4-hydroxy-3(2H)furanone.

16. The method of claim 13, wherein said analog is a compound of the formula 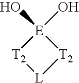
wherein E is selected from the group consisting of B, P, and S;
T₁, and T₂ are each independently selected from the group consisting of O, NR, and CH₂, where R═H or C₁-C₈ alkyl, or C₁-C₈ oxoalkyl; and
L is selected from the group consisting of ethylene, propylene, and four to six-membered alicyclic and aromatic rings;
or a pharmaceutically acceptable salt thereof.

17. The method of claim 16, wherein E is B or P; T₁ and T₂ are 0 and L is tetrahydrofuran group bearing a keto, a hydroxy, and a carboxamido functional group.

18. The method of claim 17 wherein said compound is represented by the formula

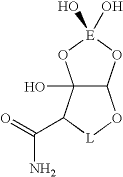

19. The method of claim 13 which further comprises administration of another anti-inflammatory agent.

20. The method of claim 19, wherein said other anti-inflammatory agent is selected from the group consisting of a corticosteroid, an NSAID and a monoclonal antibody against TNF-α.

21. A method for treating inflammation in a mammal which comprises administering autoinducer-2, an autoinducer-2 analog or an autoinducer-2 agonist to a mammal in an amount and for a time sufficient to ameliorate or reduce inflammation signalled through Toll-like receptors associated with increased iNOS activity.

22. The method of claim 21, wherein autoinducer-2 is administered.

23. The method of claim 21, wherein said analog is 5-methyl-4-hydroxy-3(2H)furanone.

24. The method of claim 21, wherein said analog is a compound of the formula 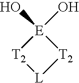
wherein E is selected from the group consisting of B, P, and S;
T₁, and T₂ are each independently selected from the group consisting of O, NR, and CH₂, where R═H or C₁-C₈ alkyl, or C₁-C₈ oxoalkyl; and
L is selected from the group consisting of ethylene, propylene, and four to six-membered alicyclic and aromatic rings;
or a pharmaceutically acceptable salt thereof.

25. The method of claim 24, wherein E is B or P; T₁ and T₂ are 0 and L is tetrahydrofuran group bearing a keto, a hydroxy, and a carboxamido functional group.
